(19) United States
(12) Patent Application Publication
(10) Pub. No.: US 2008/0172065 A1
(43) Pub. Date: Jul. 17, 2008

(54) MEDICAL DEVICE WITH BEACON

(76) Inventors: ISAAC OSTROVSKY, Wellesley, MA (US); Robert M. Rauker, Chester Springs, PA (US); William J. Shaw, Cambridge, MA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/623,875

(22) Filed: Jan. 17, 2007

(51) Int. Cl. A61B 17/22

(52) U.S. Cl. 606/113

(57) ABSTRACT

An apparatus includes an elongate body configured to be at least partially inserted into a gastrointestinal lumen. The elongate body is configured to perform a medical procedure within the gastrointestinal lumen. A beacon element is coupled to the elongate body. The beacon element is configured to be identified within the gastrointestinal lumen using a sensing device. A method includes imaging a gastrointestinal lumen with an imaging device. An apparatus is inserted at least partially into a gastrointestinal lumen. The apparatus has an elongate portion, a tool portion and a beacon element. The location of the beacon element is identified within the gastrointestinal lumen using a sensing device. The tool portion is moved to an area of interest within the gastrointestinal lumen based on the location of the beacon element.

### BACKGROUND

This invention relates to medical devices and methods, and more particularly, to an apparatus and method for maneuvering a medical tool within a gastrointestinal lumen using a radioactive element.

Colorectal cancer is one of the leading causes of deaths from malignancy in the United States, with only lung cancer causing more deaths annually. Colon cancer can be prevented because it usually begins as a benign polyp that grows slowly for several years before becoming cancerous. If polyps are detected and removed, the risk of developing colon cancer is significantly reduced.

Unfortunately, widespread colorectal screening and preventive efforts are hampered by several practical impediments, including limited resources, methodologic inadequacies, and poor patient acceptance leading to poor compliance. Moreover, some tests, such as the fecal occult blood test (FOBT) fail to detect the majority of cancers and pre-cancerous polyps. Additionally, since a sigmoidoscopy only examines a portion of the colon, it also misses many polyps that occur in the remainder of the colon. The accuracy of other tests, such as the barium enema, vary and are not always reliable.

A technique for detecting colorectal cancer using helical computed tomography (CT) to create computer simulated intraluminal flights through the colon was proposed as a novel approach for detecting colorectal neoplasms by Vining D J, Shifrin R Y, Grishaw E K, Liu K, Gelfand D W, Virtual colonoscopy (Abst), Radiology Scientific Prgm 1994; 193(P):446. This technique was first described by Vining et al. in an earlier abstract by Vining D J, Gelfand D W, Noninvasive colonoscopy using helical CT scanning, 3D reconstruction, and virtual reality (Abst), SGR Scientific Program, 1994. This technique, referred to as “virtual colonoscopy”, requires a cleansed colon insufflated with air, a helical CT scan of approximately 30 seconds, and specialized three-dimensional (3D) imaging software to extract and display the mucosal surface. The resulting endoluminal images generated by the CT scan are displayed to a medical practitioner for diagnostic purposes.

There have been several advances in virtual colonoscopy that have improved the imaging techniques, making it a more viable and effective screening option. One advantage of using a virtual colonoscopy as a screening process is the reduction of the invasiveness of a traditional colonoscopy. Traditional colonoscopies are performed using a colonoscope that has a relatively large diameter (i.e., sufficient to form a seal with the anus) that includes, among other instruments, a scope, multiple lumens for introducing gas and/or liquid, and a working channel for introducing a snare or similar device into the colon.

Another advantage of the virtual colonoscopy procedure is the elimination of the preparation process associated with a traditional colonoscopy. The typical preparation process involves the use of strong laxatives to purge any fecal waste from the colon. Such a process is extremely uncomfortable and is often cited as one of the least desirable parts of the whole procedure. Complete purging is not necessary with the virtual colonoscopy procedure. Rather, a fecal contrasting agent is used to facilitate digital subtraction of any residual feces from the virtual image.

Even though the virtual colonoscopy is largely non-invasive as a screening process, a need still exists for non-invasive and minimally invasive devices and methods for treating a gastrointestinal lumen, such as removing polyps within a colon, in the event the virtual colonoscopy, or other imaging modality, identifies a problem area within the colon.

### SUMMARY OF THE INVENTION

An apparatus includes an elongate body configured to be at least partially inserted into a gastrointestinal lumen. The elongate body is configured to perform a medical procedure within the gastrointestinal lumen. A beacon element is coupled to the elongate body. The beacon element is configured to be identified within the gastrointestinal lumen using a sensing device. A method includes imaging a gastrointestinal lumen on an imaging device. An apparatus is inserted at least partially into the gastrointestinal lumen. The apparatus has an elongate portion, a tool portion and a beacon element. The location of the beacon element is identified within the gastrointestinal lumen using a sensing device. The tool portion is moved to an area of interest within the gastrointestinal lumen based on the location of the beacon element. Such a medical device can, for example, eliminate the need for prolonged radiation exposure during use and tracking of the medical device.

### BRIEF DESCRIPTION OF THE DRAWINGS

The present invention is described with reference to the accompanying drawings.

FIG. 1 is FIG. 1 is an illustration of a large intestine.

FIGS. 2A and 2B illustrate different types of polyps in a colon.

FIG. 3 is a schematic illustration of an apparatus according to an embodiment of the invention.

FIG. 4 is a schematic illustration of a system according to an embodiment of the invention.

FIG. 5 is a side view of an apparatus according to an embodiment of the invention.

FIG. 6 is a side view of an apparatus according to an embodiment of the invention.

FIG. 7 is a side view of an apparatus according to an embodiment of the invention.

FIG. 8 is a side view of an apparatus according to another embodiment of the invention.

FIG. 9 is a side view of an apparatus according to another embodiment of the invention shown partially in cross-section and in a first position.

FIG. 10 is a side view of the apparatus of FIG. 9 shown partially in cross-section and in a second position.

FIG. 11 is a flowchart of a method according to an embodiment of the invention.

### DETAILED DESCRIPTION

An apparatus includes an elongate body configured to be at least partially inserted into a gastrointestinal lumen. The elongate body is configured to perform a medical procedure within the gastrointestinal lumen. A beacon element, such as a radioactive element or an electromagnetic radiator, is coupled to the elongate body. The beacon element is configured to be identified within the gastrointestinal lumen using a sensing device. A method includes imaging a gastrointestinal lumen on an imaging device. An apparatus is inserted at least partially into the gastrointestinal lumen. The apparatus has an elongate portion, a tool portion and a beacon element. The location of the beacon element is identified within the gastrointestinal lumen using a sensing device. The tool portion is moved to an area of interest within the gastrointestinal lumen based on the location of the beacon portion

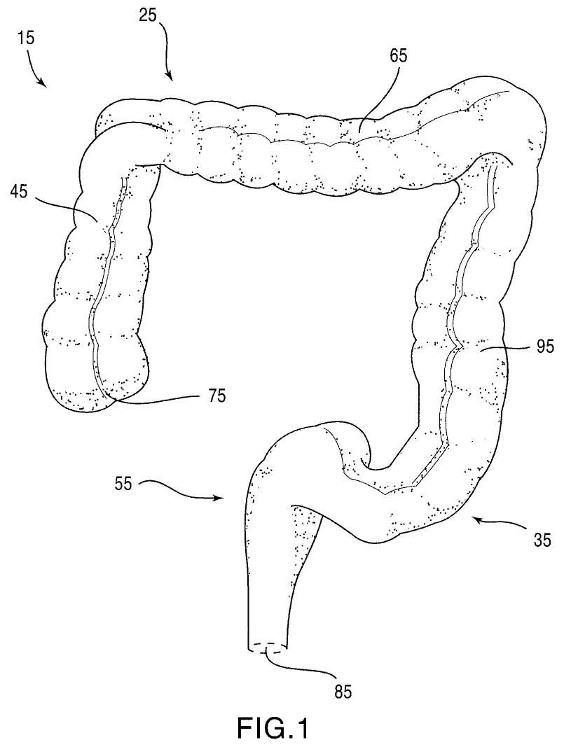

An illustration of a large intestine (also called the large bowel) 15 is provided in FIG. 1 by way of background and reference. The colon 25 is the longest part of the large intestine 15, which is a tube-like organ connected to the small intestine (not illustrated) at one end and the anus 85 at the other. The colon 25 and the rectum 55 form the large intestine 15. The colon 25 is the first 4 to 5 feet of the large intestine 15, and the rectum 55 is the last 4 to 5 inches. The part of the colon 25 that joins to the rectum 55 is called the sigmoid colon 35. The junction of the two parts is often referred to as the rectosigmoid colon or rectosigmoid process. The part of the colon 25 that joins to the small intestine is called the cecum 75. The cecum 75 is adjacent the ascending colon 45, which is connected to the transverse colon 65. The transverse colon 65 is connected to the descending colon 95, which is connected to the sigmoid colon 35. The colon 25 removes/absorbs water and some nutrients and electrolytes from partially digested food. The remaining material, solid waste, called stool or feces, moves through the colon 25 to the rectum 55 and leaves the body through the anus 85.

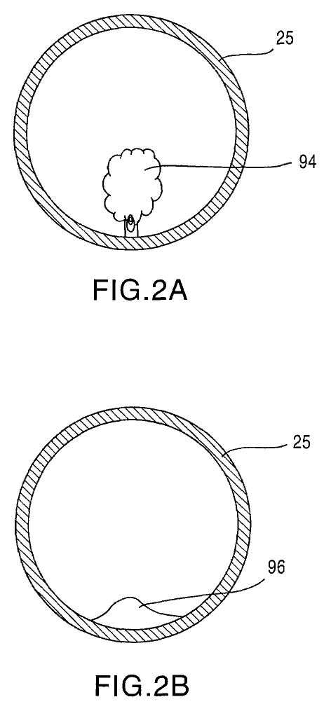

FIGS. 2A-2B illustrate various types of polyps that can form in the colon. A gastrointestinal polyp is a mass of the mucosal surface of the intestine that protrudes into the passageway of the bowel. Polyps can be neoplastic, non-neoplastic, or submucosal. Adenomatous polyps are abnormal growths in the colon and are more likely to develop into or already contain cancer than other types of colon polyps. Adenomatous polyps, however, usually contain tissue that is abnormal but not necessarily cancerous, hence the importance of being able to completely remove a polyp from the colon. The size, type of tissue, and degree of abnormality (mild, moderate, or severe) in a polyp determines the likelihood that it contains cancer.

Some adenomatous polyps are attached to the wall of the colon 25 (or rectum) by a stalk (a pedunculated polyp 94) as illustrated in FIG. 2A. Some polyps have a broad base with little or no stalk (a sessile polyp 96) as illustrated in FIG. 2B.

The apparatuses, systems and methods of the present invention involve the use of an apparatus in conjunction with known imaging devices, including virtual imaging modalities, to maneuver the apparatus through a gastrointestinal lumen, such as a colon. Although the below description focuses primarily on medical treatments within a colon, such as removal of a polyp from a colon, the methods and medical procedures described can be used in other gastrointestinal lumens.

Apparatuses and methods are provided that combine visual techniques, such as X-rays, with information that can be produced by a sensor moving through anatomy of a patient to produce a three dimensional computer model of the anatomy. For example, a computed tomography (CT) scan can generate cross-sectional images every 2-8 millimeters of its longitudinal movement. By scanning along human anatomy, for example, within a colon, the cross-sectional images of the colon and surrounding organs can be taken. A sensor can be configured to move along the same path as the scanning to generate data specifying a location of the sensor. The sensor information and scanning information can be combined by stacking cross-sectional images along the sensor's path, and a three dimensional representation of the anatomy can be modeled.

The above described sensor can be, for example, an electromagnetic receiver as described in U.S. Pat. No. 6,232,476, the disclosure of which is hereby incorporated by reference in its entirety. Electromagnetic transmitters can be placed within the anatomy of a patient, and used in conjunction with the receivers to generate specific locations of the transmitters within the anatomy. In some embodiments, the transmitter is a radiator or radioactive element. For example, the radioactive element can be a gamma radiator or a beta radiator. Radioactive sensors (e.g., receivers) can identify the radiation associated with the radioactive element and produce location data corresponding to the spatial location of the radioactive element.

A computerized three-dimensional model can then be produced that is a virtual representation of the anatomy (e.g., colon). A physician can use the model to determine, for example, if there are polyps present that need to be removed. If further treatment is needed (e.g., a polyp is identified), a medical device such as a snare having a beacon element as described in more detail below can be inserted into the patient's body. The medical device can be advanced within the body lumen (e.g., colon) and the location of the beacon element can be registered and identified on the three-dimensional model of the colon on a computer. This procedure allows the physician to track, locate, or direct the medical device within the body lumen without the use, for example, of an X-ray or colonoscope.

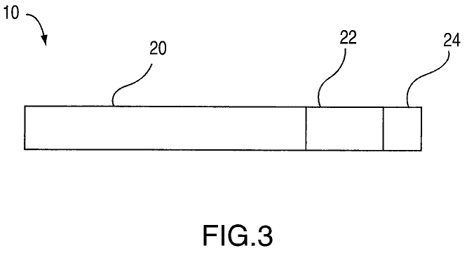

FIG. 3 is a schematic illustration of an apparatus of the invention. The apparatus 10 includes an elongate body 20 having a tool portion 22, and a beacon element 24. The elongate body 20 and the tool portion 22 can include any known configuration for a medical device. For example, the elongate body 20 can include a snare configuration used to sever and remove tissue from within a body lumen, such as a polyp within a gastrointestinal lumen. The elongate body 20 can also include other devices such as forceps, graspers, needles, cannulas, delivery devices, etc.

A beacon element 24 can be disposed on the elongate body 20 in a variety of different configurations that will be discussed in more detail below. The beacon element 24 can be, for example, a radioactive sensor or an electromagnetic sensor, configured to be identified using a sensing device, (e.g., a receiver). The sensing device can in some embodiments be associated with an imaging device 26. The beacon element 24 can in some embodiments, include radio waves, light waves, or radio frequency identification (RFID), etc. The beacon element 24 can be passive (e.g., detection of electromagnetic radiation), or active (e.g., activated RFID). The beacon element 24 can be, for example, a gamma emitter or a beta emitter, that is visible on a CT device. The corresponding sensing device can also be active or passive depending on the type of beacon element 24.

The imaging device 26 can include a variety of different imaging modalities, such as a CT device, a magnetic resonance imaging (MRI) device, or an X-ray device. A virtual imaging modality can also be used, such as a virtual colonoscopy, or any other type of non-invasive imaging modality. The imaging device 26 can include a receiver for receiving a signal associated with the beacon element 24.

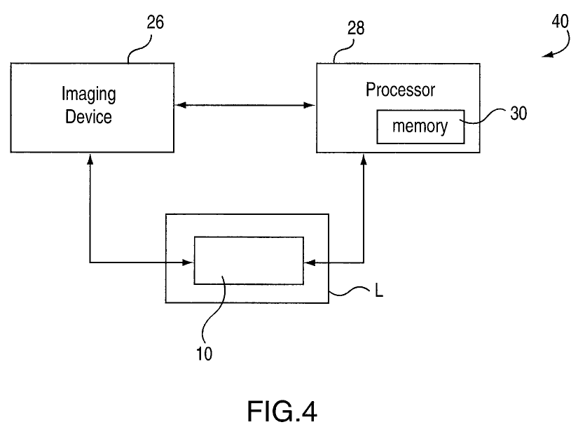

The apparatus 10 and the imaging device 26 can be in communication with a processor 28, as shown in FIG. 4. FIG. 4 is a schematic illustration of a system 40 that includes the imaging device 26, the processor 28, and the apparatus 10 positioned within a gastrointestinal lumen L. The imaging device 26 can send, transfer or otherwise provide imaging data to the processor 28. The imaging data can include, for example, an image of the gastrointestinal lumen and data associated with the beacon element 24. The processor 28 can use the image data to create a model of the gastrointestinal lumen. The model can be used to assist in accurately identifying the location of the beacon element 24, without the use of external radiation, and thus the location of the elongate body 20, within the gastrointestinal lumen at a given time.

The processor 28 can be, for example, a commercially available personal computer, or a less complex computing or processing device that is dedicated to performing one or more specific tasks. The processor 28, according to one or more embodiments of the invention, can be a commercially available microprocessor. Alternatively, the processor 28 can be an application-specific integrated circuit (ASIC) or a combination of ASICs, which are designed to achieve one or more specific functions, or enable one or more specific devices or applications. In yet another embodiment, the processor 28 can be an analog or digital circuit, or a combination of multiple circuits.

The processor 28 can include a memory component 30. The memory component 30 can include one or more types of memory. The processor 28 can store data in the memory component 30 or retrieve data previously stored in the memory component 30. The components of the processor 28 can communicate with devices external to the processor 28 by way of an input/output (I/O) component (not shown). According to one or more embodiments of the invention, the I/O component can include a variety of suitable communication interfaces.

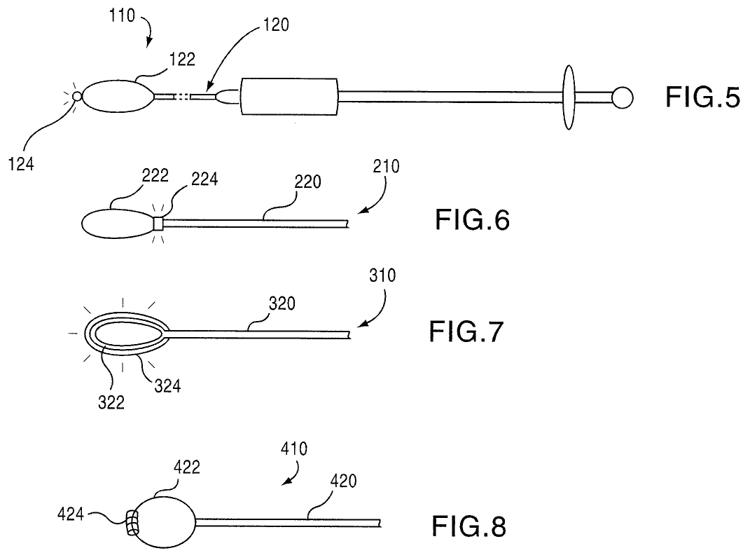

FIG. 5 is a side view of an apparatus according to an embodiment of the invention. The apparatus 110 includes an elongate body 120 having a tool portion 122 and a beacon element 124. In this embodiment, the beacon element 124 is disposed on a distal end of the tool portion 122.

FIG. 6 is a side view of an apparatus according to an embodiment of the invention. The apparatus 210 includes an elongate body 220 having a tool portion 222 and a beacon element 224. In this embodiment, the beacon element 224 is disposed at a proximal end of the tool portion 222.

In some embodiments, a radioactive element can be disposed along at least a portion of a perimeter of a tool portion. For example, FIG. 7 is a side view of an apparatus according to another embodiment of the invention. The apparatus 310 includes an elongate body 320 having a tool portion 322 and a beacon element 324. In this embodiment, the beacon element 324 is disposed substantially along a perimeter of the tool portion 322. In some embodiments, a radioactive element can be disposed along only a portion of a perimeter of a tool portion, as shown in FIG. 8. FIG. 8 illustrates an apparatus 410 that includes an elongate body 420 having a tool portion 422 and a beacon element 424.

In any of the embodiments described above, the beacon element can be coupled to the tool portion, by a variety of different attachment or coupling methods. For example the beacon element can be crimped, coiled, banded, glued, melted on, wrapped, coupled by heat shrinking, swaged (e.g., compressing a beacon element on to the elongate body), etc. to the elongate body.

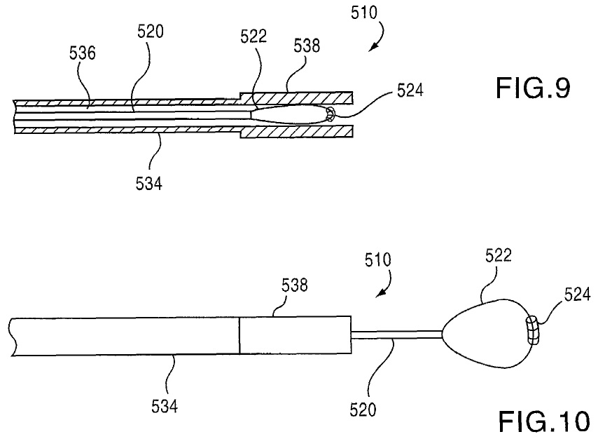

An apparatus according to any of the above-described embodiments can also be used in conjunction with a sheath or sleeve member as shown, for example, in FIGS. 9 and 10. FIGS. 9 and 10 illustrate an apparatus 510 that includes an elongate body 520 having a tool portion 522 and a beacon element 524 disposed at the tool portion 522. The apparatus 510 also includes a sleeve 534 that defines a lumen 536 in which the elongate body 520 can be movably disposed as shown in FIG. 9. The sleeve 534 can be formed with any suitable material used for medical devices, including for example, a polymer. In some embodiments, the sleeve 534 can also include a tungsten portion 538 that substantially covers the tool portion 522 and/or the beacon element 524 when the elongate body is disposed within the lumen 536 of the sleeve 534. Other suitable cannulas or sleeve members can alternatively be used.

In use, the apparatus 510 can be positioned in a desired location within a patient's body with the tool portion 522 of the elongate body 520 disposed within the lumen 536 of the sleeve 534. The sleeve 534 can then be pulled proximally such that the tool portion 522 of the elongate body 520 is disposed outside of the lumen 536 and the beacon element 524 is uncovered.

The above-described embodiments are merely examples of possible configurations for an apparatus of the invention. For example, one or more beacon elements can be disposed at any location along an elongate body of the apparatus. In addition, an apparatus can embody a variety of different configurations for a medical device. Although not specifically illustrated, a sheath or sleeve, such as sleeve 536 described above, can be included in any of the above-described embodiments.

The various components of an apparatus according to the invention can be formed with suitable materials used for such medical uses. For example, the elongate body and sleeve can be formed with various plastics, such as polymers, or metals such as stainless steel. The beacon element can be formed with various different types of materials as described previously.

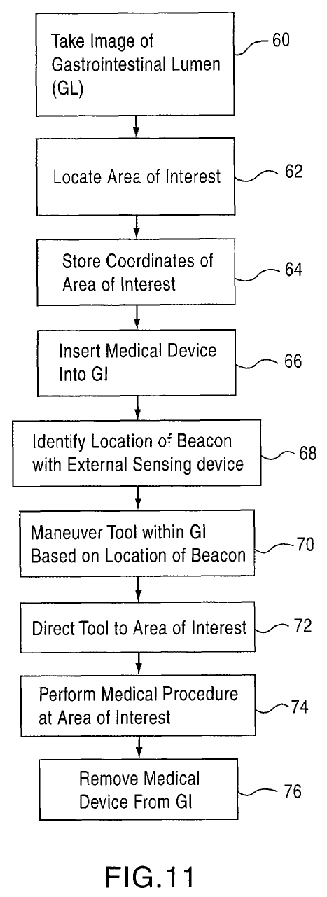

A method of using an apparatus (also referred to as a medical device) according to an embodiment of the invention is illustrated in a flowchart in FIG. 11. At 60, an image of a body lumen, such as a gastrointestinal lumen (e.g., a colon), is taken. An area of interest (e.g., a lesion) is identified at 62. Coordinates of the area of interest are stored within a processor at 64. At 66, a medical device 10 (110, 210, 310) having an elongate body, a tool portion and a beacon element, is inserted at least partially into the gastrointestinal lumen. At 68, a location of the beacon element is identified within the gastrointestinal lumen using an external sensing device (e.g., a passive or active sensing device). At 70, the tool can be maneuvered or directed within the gastrointestinal lumen based on the location of the beacon element (e.g., by viewing on the beacon element on the imaging device). At 72, the tool can be directed to the area of interest. At step 74, a medical procedure is performed on the area of interest, such as snaring a polyp at the area of interest. The medical device is removed from the gastrointestinal lumen, at step 76.

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

The previous description of the various embodiments of an apparatus is provided to enable any person skilled in the art to make or use the invention. While the invention has been particularly shown and described with reference to embodiments thereof, it will be understood by those skilled in art that various changes in form and details may be made.

For example, although described as being used in a medical procedure performed in a gastrointestinal lumen, an apparatus as described herein can be used for other medical procedures in other areas of a patient's body. An apparatus can be a variety of different configurations, shapes and/or sizes and be formed with various different materials not specifically described. An apparatus can include various combinations and sub-combinations of the embodiments described herein. For example, a beacon can be a variety of different materials that are detectable on a sensing device and be a variety of different shapes and sizes. The elongate body can also be a variety of different shapes, sizes and configurations and can include many different types of medical devices.

What is claimed is:

1. An apparatus, comprising:
an elongate body configured to be at least partially inserted into a gastrointestinal lumen, the elongate body configured to perform a medical procedure within the gastrointestinal lumen; and
a beacon element coupled to the elongate body, the beacon element configured to be identified within the gastrointestinal lumen using a sensing device.

2. The apparatus of claim 1, wherein the beacon element is active.

3. The apparatus of claim 1, wherein the beacon element is passive.

4. The apparatus of claim 1, wherein the sensing device is active.

5. The apparatus of claim 1, wherein the sensing device is passive.

6. The apparatus of claim 1, wherein the gastrointestinal lumen is a colon, the elongate body includes a snare portion, the snare portion configured to capture a polyp from within the colon.

7. The apparatus of claim 1, further including a sheath coupled to the elongate body.

8. The apparatus of claim 1, wherein the sensing device includes a sensor, the beacon element configured to be detected by the sensor to identify a location of the beacon element within the gastrointestinal lumen.

9. The apparatus of claim 1, wherein the beacon element is a gamma emitter.

10. The apparatus of claim 1, wherein the sensing device is associated with an imaging device.

11. The apparatus of claim 1, wherein the sensing device is associated with a computed tomography scanner.

12. The apparatus of claim 1, wherein the beacon element is radioactive.

13. The apparatus of claim 1, wherein the beacon element includes a radio frequency identifier.

14. The apparatus of claim 1, wherein the sensing device is configured to receive a radio frequency identification signal from the beacon element.

15. A method, comprising:
imaging a gastrointestinal lumen on an imaging device;
inserting an apparatus at least partially into the gastrointestinal lumen, the apparatus having an elongate portion, a tool portion and a beacon portion;
identifying a location of the beacon portion within the gastrointestinal lumen using a sensing device; and
moving the tool portion to an area of interest within the gastrointestinal lumen while monitoring the location of the beacon element on a sensing device.

16. The method of claim 15, wherein the gastrointestinal lumen is a colon, the method further comprising:
snaring a polyp from within the colon using the tool portion; and
withdrawing the apparatus from the colon.

17. The method of claim 15, wherein the gastrointestinal lumen is a colon.

18. The method of claim 15, further comprising:
prior to the identifying, locating an area of interest within the gastrointestinal lumen.

19. The method of claim 15, further comprising:
prior to the identifying, locating an area of interest within the gastrointestinal lumen; and
storing coordinates associated with the area of interest.

20. An apparatus, comprising:
an elongate body including a proximal end portion and a distal end portion, the distal end portion of the elongate body configured to be inserted at least partially in a colon; and
a medical tool, the medical tool coupled to the elongate body, the medical tool including a beacon portion configured to be identified within the colon using a sensing device.

21. The apparatus of claim 20, wherein the sensing device is associated with an imaging device.

22. The apparatus of claim 20, wherein the beacon portion is a gamma emitter.

23. The apparatus of claim 20, wherein the medical tool is configured to capture a polyp from within the colon.

24. The apparatus of claim 20, wherein the medical tool is configured to view an interior of the colon.

25. The apparatus of claim 20, wherein the beacon element is an electromagnetic radiator.

26. The apparatus of claim 20, wherein the beacon element is radioactive.

27. The apparatus of claim 20, wherein the beacon element includes a radio frequency identifier.

28. The apparatus of claim 20, wherein the sensing device is configured to receive a radio frequency identification signal from the beacon element.
